(19) United States
(12) Patent Application Publication
(10) Pub. No.: US 2012/0295054 A1
(43) Pub. Date: Nov. 22, 2012

(54) TAPE HAVING PATTERN OF ADHESIVE LAYERS

(75) Inventor: John A. HOPE, Warminster, PA (US)

(73) Assignee: PERFORMTEX PRODUCTS LLC, Warminster, PA (US)

(21) Appl. No.: 13/111,203

(22) Filed: May 19, 2011

(51) Int. Cl. C09J 7/02; A61L 15/58; B32B 3/10

(52) U.S. Cl. 428/41.8; 428/196; 428/195.1

(57) ABSTRACT

An adhesive tape includes a base material and a plurality of adhesive layers disposed on a first side of the base material. A plurality of exposed base material areas separate the plurality of adhesive layers, such that when the adhesive tape is viewed from the first side, the plurality of exposed base material areas appear to be a plurality of intersecting lines.

### FIELD OF INVENTION

The present disclosure relates to the field of adhesive tape. More particularly, the present disclosure relates to a body-adhesive tape that is adhered directly to the skin.

### BACKGROUND

Body-adhesive tape is used for medical, athletic, therapeutic, or other purposes. Known body-adhesive tape includes a base material such as cloth, elastic, paper, or a combination thereof. An adhesive layer is disposed on one side of the base material. In some known embodiments, the adhesive layer is continuous and covers the entire base material. In other embodiments, the adhesive layer is disposed in a pattern or is randomly disposed about the base material.

A removable release liner is provided on some known body-adhesive tapes. A release agent may also be disposed on the base material. Known body-adhesive tapes are provided in rolls, strips, or pre-cut patterns.

### SUMMARY OF THE INVENTION

In one embodiment, a body-adhesive tape includes a base cloth having a plurality of elastic threads and a plurality of non-elastic threads. A plurality of adhesive layers are disposed on a first side of the base cloth, including a plurality of elongated adhesive layers and a plurality of non-elongated adhesive layers. A plurality of exposed cloth areas separate the plurality of adhesive layers. The plurality of exposed cloth areas are a form of a plurality of intersecting lines.

In an alternative embodiment, an adhesive tape includes a base material and an adhesive layer disposed on a first side of the base material. A plurality of gaps are disposed in the adhesive layer, separating the adhesive layer and defining a plurality of elongated adhesive portions and a plurality of non-elongated adhesive portions, arranged in a selected pattern. The adhesive tape has a first level of permeability at portions of the base material on which the adhesive layer is disposed, and a second level of permeability at the plurality of gaps, the second level of permeability being higher than the first level of permeability.

In another alternative embodiment, an adhesive tape includes a base material and a plurality of adhesive layers disposed on a first side of the base material. A plurality of exposed base material areas separate the plurality of adhesive layers, such that when the adhesive tape is viewed from the first side, the plurality of exposed base material areas appear to be a plurality of intersecting lines.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 is a perspective view of one embodiment of a roll of adhesive tape;

FIG. 2 is a side view of one embodiment of the adhesive tape;

FIG. 3 is a front plan view of a portion of the adhesive tape;

FIG. 4 is a detailed front plan view of a portion of the adhesive tape;

FIG. 5 is a front plan view of an alternative embodiment of adhesive tape; and

FIG. 6 is a front plan view of another alternative embodiment of adhesive tape.

### DETAILED DESCRIPTION

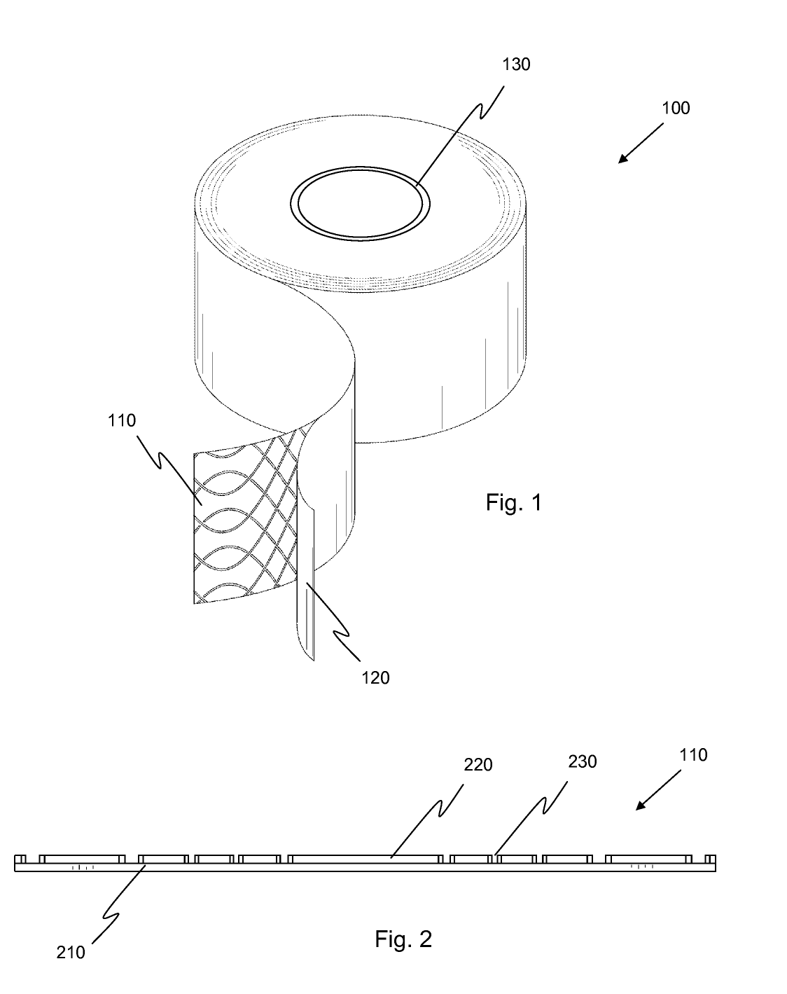

FIG. 1 illustrates a perspective view of one embodiment of a roll 100 of adhesive tape 110. The roll 100 includes the adhesive tape 110 and a removable release liner 120. The removable release liner 120 may be a paper or plastic based material. In one embodiment, the removable release liner 120 is coated on one or two sides with a release agent, which provides a release effect against the adhesive tape 110. Exemplary release agents include silicone and other materials having a low surface energy. In an alternative embodiment (not shown), the roll 100 does not include a removable release liner, but is instead rolled directly onto itself.

The roll 100 further includes a central member 130, about which the adhesive tape 110 and the removable release liner 120 are wrapped. In one embodiment, the central member 130 is a cardboard tube. In an alternative embodiment, the central member 130 is a polymeric tube or ring. In another alternative embodiment, no central member is employed.

FIG. 2 illustrates a detailed side view of one embodiment of adhesive tape 110. The adhesive tape 110 includes a base material 210. The base material 210 may be paper, plastic film, cloth, or metal foil. In one embodiment, the base material is treated with a primer or a release agent (not shown). The primer or release agent may be silicone, and may be applied in the form of a water-based or oil-based spray or resin. In another embodiment, the base material is treated with a water-repellant treatment. The water-repellant treatment may be a polymeric resin or spray.

In one embodiment, the base material 210 is a cloth having weft threads and warp threads. The weft threads include a plurality of non-elastic threads. The warp threads include a plurality of elastic threads and a plurality of non-elastic threads. For example, the elastic threads may be twisted with the non-elastic threads. Exemplary elastic threads include, without limitation, rubberized threads, synthetic threads, and wool threads. Exemplary non-elastic threads include cotton.

In an alternative embodiment, the weft threads also include elastic threads. In another alternative embodiment, the warp threads include only elastic threads. In another alternative embodiment, all of the threads are non-elastic.

An adhesive layer 220 is disposed on a first side of the base material 210. In one embodiment, the adhesive layer 220 is a pressure-sensitive adhesive layer. In one specific embodiment, the adhesive layer 220 is an acrylic-based, pressure-sensitive adhesive layer. Alternatively, synthetic rubber or natural rubber based adhesive may be used. Non-pressure-sensitive adhesive layers may also be employed. For example, contact adhesives, drying adhesives, and hot adhesives may also be employed. It should be understood that the adhesive layer 220 is not limited to any particular type of adhesive.

A plurality of gaps 230 are disposed in the adhesive layer 220, separating the adhesive layer 220 into a plurality of adhesive layers arranged in a selected pattern. The gaps 230 expose the base material 210, and so may be referred to as exposed base material areas or exposed cloth areas separating the plurality of adhesive layers 220.

The adhesive tape 110 has a first level of permeability at portions of the base material 210 on which the adhesive layers 220 are disposed, and a second level of permeability at the gaps 230. The second level of permeability is higher than the first level of permeability.

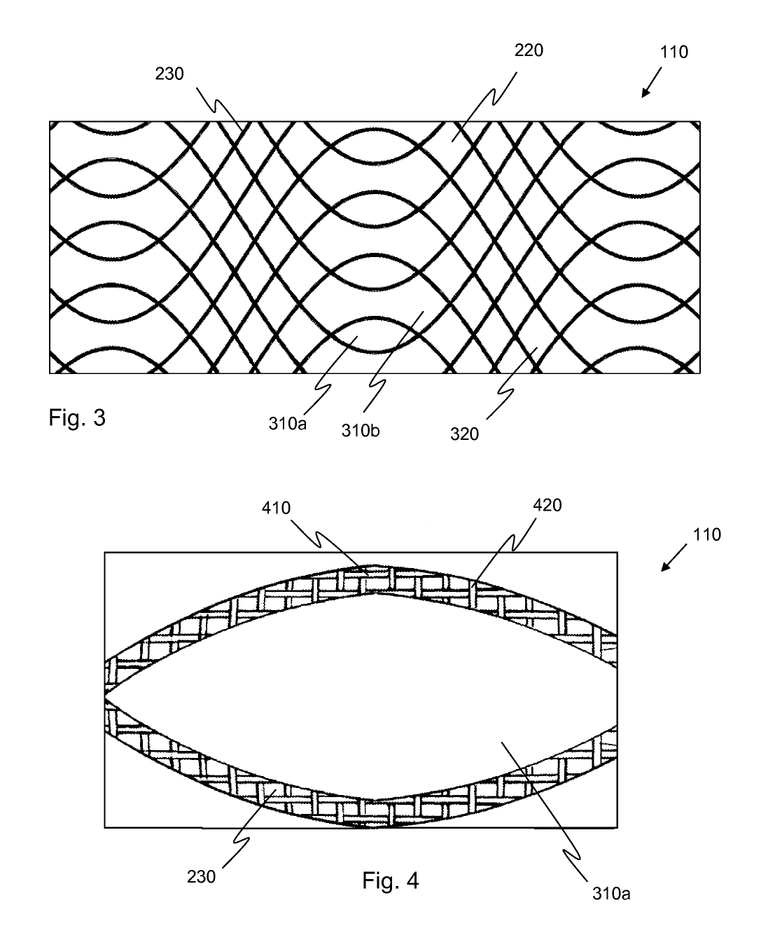

FIG. 3 illustrates a front plan view of a portion of adhesive tape 110. In this embodiment, the exposed cloth areas 230 are in the form of a plurality of intersecting lines when viewed from the front. In the illustrated embodiment, the plurality of intersecting lines are a plurality of substantially sinuous lines. However, it should be understood that other types of curved intersecting lines may be employed. In an alternative embodiment (not shown), the exposed cloth areas are in the form of a plurality of intersecting straight lines when viewed from the front.

The plurality of intersecting lines separate the adhesive layer 220 into a plurality of elongated adhesive layers 310 and a plurality of non-elongated adhesive layers 320. In the illustrated embodiment, the plurality of non-elongated adhesive layers 320 are diamond-shaped adhesive portions. The plurality of elongated adhesive layers 310 include a plurality of biconvex lens-shaped adhesive portions 310a and a plurality of biconcave lens-shaped adhesive portions 320b. The biconvex lens-shaped adhesive portions 320a have ogive ends. For purposes of this disclosure, an “ogive” is a bullet-shaped intersection of two curves that have substantially the same radius of curvature. The biconcave lens-shaped adhesive portions 320b also have ogive ends.

In alternative embodiments (not shown), the exposed base material areas may be in the form of a plurality of intersecting straight lines. In another alternative embodiment, the exposed cloth areas may be in the form of a plurality of intersecting straight and curved lines. In yet another alternative embodiment, the exposed cloth areas may be in the form of non-intersecting lines. It should be understood that different patterns of exposed base material areas will result in different patterns of adhesive layers.

FIG. 4 illustrates a detailed front plan view of a portion of adhesive tape 110, showing a biconvex lens-shaped adhesive portion 320a. As can be seen in this view, the gaps 230 expose the underlying base material 210. In this embodiment, the base material 210 is a cloth having warp threads 410 and waft threads 420 that are exposed.

Because the adhesive tape 110 has a higher permeability at the exposed base material areas 230, such tape may be suitable as a body-adhesive tape. Sweat or other moisture on the body may evaporate through the higher permeability areas of the adhesive tape 110, thus preventing skin macerations. Additionally, the adhesive tape described herein may be used as kinesiology therapeutic tape. Kinesiology therapeutic tape is used in therapy to relax overused muscles and in rehabilitation to facilitate underused muscles. The pattern on the tape's adhesive may have a lifting effect on the skin which can reduce swelling and inflammation by improving circulation and reduce pain by taking pressure off pain receptors. The elastic properties of the tape may also be utilized in a mechanical fashion to limit or encourage a specific motion at a joint. However, it should be understood that such possible benefits are not part of the claimed invention. Moreover, the adhesive tape disclosed herein is not limited to kinesiology therapeutic tape or body-adhesive tape.

While the adhesive tape 110 is shown in FIG. 1 as being disposed on a roll 100, it should be understood that the adhesive tape need not be provided in a roll. In an alternative embodiment, the adhesive tape may be provided in strips. For example, the front plan view of a portion of adhesive tape of FIG. 3 may be a view of a strip of adhesive tape, rather than a portion of a roll.

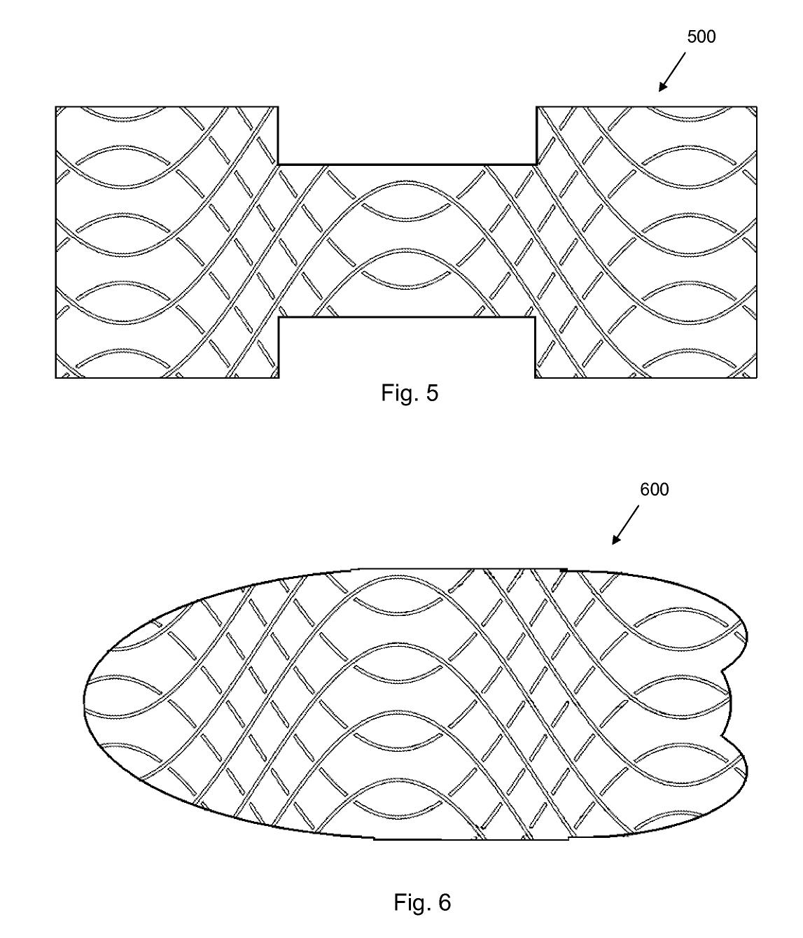

The adhesive tape may also be provided in pre-cut patterns. For example, FIG. 5 illustrates a front plan view of an alternative embodiment of adhesive tape 500. In this embodiment, the adhesive tape 500 is provided in an elongated H-shape. Additionally, FIG. 6 illustrates a front plan view of another alternative embodiment of adhesive tape 600. In this embodiment, the adhesive tape 600 has a plurality of rounded sides.

The pre-cut patterns of FIGS. 5 and 6 should be understood to be merely exemplary. The adhesive tape may be provided in any desired pattern without departing from the scope of this invention.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present disclosure has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the disclosure, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A body-adhesive tape comprising:
a base cloth having a plurality of elastic threads and a plurality of non-elastic threads;
a plurality of adhesive layers disposed on a first side of the base cloth, including a plurality of elongated adhesive layers and a plurality of non-elongated adhesive layers; and
a plurality of exposed cloth areas separating the plurality of adhesive layers, the plurality of exposed cloth areas being in a form of a plurality of intersecting lines.

2. The body-adhesive tape of claim 1, wherein the plurality of intersecting lines are a plurality of substantially sinuous lines.

3. The body-adhesive tape of claim 1, further comprising a release agent layer.

4. The body-adhesive tape of claim 1, further comprising a removable release liner.

5. The body-adhesive tape of claim 1, wherein the base cloth is formed of a plurality of warp threads and a plurality of weft threads.

6. The body-adhesive tape of claim 5, wherein the plurality of warp threads include the plurality of elastic threads.

7. The body-adhesive tape of claim 5, wherein the plurality of weft threads include the plurality of non-elastic threads.

8. The body-adhesive tape of claim 1, wherein the body-adhesive tape is provided in a roll.

9. The body-adhesive tape of claim 1, wherein the body-adhesive tape is provided in a strip.

10. The body-adhesive tape of claim 1, wherein the body-adhesive tape is provided in a pre-cut pattern.

11. An adhesive tape comprising:
a base material;
an adhesive layer disposed on a first side of the base material; and
a plurality of gaps in the adhesive layer, separating the adhesive layer and defining a plurality of elongated adhesive portions and a plurality of non-elongated adhesive portions, arranged in a selected pattern,
wherein the adhesive tape has a first level of permeability at portions of the base material on which the adhesive layer is disposed, and a second level of permeability at the plurality of gaps, the second level of permeability being higher than the first level of permeability.

12. The adhesive tape of claim 11, wherein the plurality of non-elongated adhesive portions include diamond-shaped adhesive portions.

13. The adhesive tape of claim 11, wherein the plurality of elongated adhesive portions include a plurality of biconcave lens-shaped adhesive portions.

14. The adhesive tape of claim 13, wherein each of the plurality of biconcave lens-shaped adhesive portions have ogive ends.

15. The adhesive tape of claim 11, wherein the plurality of elongated adhesive portions include a plurality of biconvex lens-shaped adhesive portions.

16. The adhesive tape of claim 15, wherein each of the plurality of biconvex lens-shaped adhesive portions have ogive ends.

17. An adhesive tape comprising:
a base material;
a plurality of adhesive layers disposed on a first side of the base material; and
a plurality of exposed base material areas separating the plurality of adhesive layers, wherein when the adhesive tape is viewed from the first side, the plurality of exposed base material areas appear to be a plurality of intersecting curved lines.

18. The adhesive tape of claim 17, wherein the plurality of exposed base material areas separate the plurality of adhesive layers into a plurality of elongated adhesive layers and a plurality of non-elongated adhesive layers.

19. The adhesive tape of claim 17, wherein the adhesive tape is a kinesiology therapeutic tape.

20. The adhesive tape of claim 17, wherein the base material is a cloth having warp threads and weft threads.
